(19) United States
(12) Patent Application Publication
(10) Pub. No.: US 2026/0215684 A1
(43) Pub. Date: Jul. 30, 2026

(54) OPHTHALMIC MICROSCOPE WITH INTEGRATED VESSEL DETECTION

(71) Applicant: Alcon Inc., Fribourg (CH)

(72) Inventors: Gangjun Liu, Portland, OR (US); Lingfeng Yu, Rancho Santa Margarita, CA (US); Sumit Paliwal, Irvine, CA (US); Luyao Ma, Fort Worth, TX (US); Paul R. Hallen, Colleyville, TX (US)

(21) Appl. No.: 19/447,575

(22) Filed: Jan. 13, 2026

Related U.S. Application Data
(60) Provisional application No. 63/751,320, filed on Jan. 30, 2025.

(51) Int. Cl. A61B 3/13; A61B 3/00; A61B 5/1455

(52) U.S. Cl. CPC A61B 3/13; A61B 3/0041; A61B 5/14555

(57) ABSTRACT

A system includes an ophthalmic microscope configured to capture video of an ophthalmic treatment. A computer system is coupled to the ophthalmic microscope and is configured to: receive a first image from the ophthalmic microscope for a first wavelength band having a first width of less than 20 nanometers; receive a second image from the ophthalmic microscope for a second wavelength band having a second width of less than 20 nanometers; generate a vessel map of a retina of the eye of the patient according to the first image and the second image, the vessel map including representations of blood vessels of the retina; and display the vessel map on a display device. The computer system may further receive a reference image for an isosbestic wavelength band of oxygenated and de-oxygenated hemoglobin and generate the vessel map according to the reference image.

### BACKGROUND

The present disclosure relates generally to providing imaging during ophthalmic surgery

The retina is permeated with a plurality of blood vessels. There are various pathologies that may affect the vessels of the retina and that may be corrected by treatment of the blood vessels. For example, the vessels may be occluded, become excessively dilated, become twisted (“tortuous”), and/or become too narrow. Abnormal vessels may grow due to diseases such as diabetic retinopathy, retinopathy of prematurity, and/or choroidal neovascularization. Various treatments may be performed to repair such conditions, such as retinal laser photocoagulation.

It would be an advancement in the art to facilitate the visualization of blood vessels of the retina, particularly during ophthalmic treatments.

### SUMMARY

In certain embodiments, a system includes an ophthalmic microscope configured to capture video of an ophthalmic treatment. A computer system is coupled to the ophthalmic microscope and is configured to: receive a first image from the ophthalmic microscope for a first wavelength band having a first width of less than 20 nanometers; receive a second image from the ophthalmic microscope for a second wavelength band having a second width of less than 20 nanometers; generate a vessel map of a retina of the eye of the patient according to the first image and the second image, the vessel map including representations of blood vessels of the retina; and display the vessel map on a display device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing ophthalmic treatments in accordance with certain embodiments.

FIG. 2 illustrates an image of a retina showing vasculature thereof.

FIG. 3 is a plot showing the absorption spectrum of oxygenated and deoxygenated blood in accordance with certain embodiments.

FIG. 4 is a schematic diagram of an imaging system in accordance with certain embodiments.

FIG. 5 is a process flow diagram of a method for visualizing retinal vasculature in accordance with certain embodiments.

FIGS. 6A to 6D illustrate the processing of images to generate a vessel map in accordance with certain embodiments.

FIG. 7 is a schematic diagram illustrating a system for visualizing retinal vasculature in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

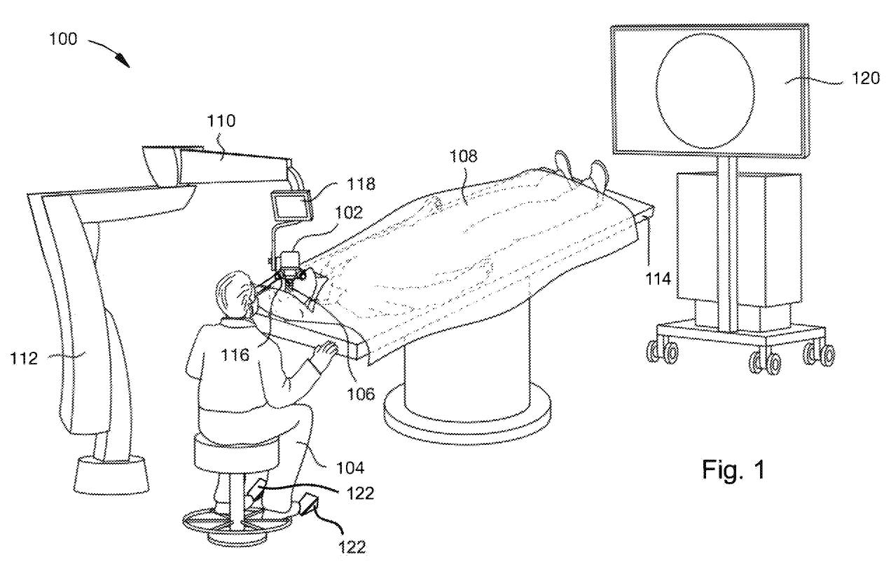

FIG. 1 illustrates an example system 100 that may be used for performing ophthalmic treatments. The system 100 includes an ophthalmic microscope 102. A surgeon 104 uses the ophthalmic microscope 102 to visualize structures on and in an eye 106 of a medical patient 108 undergoing a surgery. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing ophthalmic microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices viewable through binocular eyepieces 116 and that display images of the patient’s eye 106 that are captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively be displayed on one or more display devices. For example, the one or more display devices may include a display device 118 fastened to the supporting arm 110 above the ophthalmic microscope 102.

In order to relieve the surgeon 104 from the need to constantly look into the eye pieces 116 to obtain a stereoscopic view, the one or more display devices may include a display device 120 may be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

Operation of the ophthalmic microscope 102, surgical instruments (e.g., phaco-vit tool such as the ALCON CENTURION), and/or information displayed on the display devices 118, 120 may be controlled using foot pedals 122 operatively coupled to the ophthalmic microscope 102 and/or display devices 118, 120.

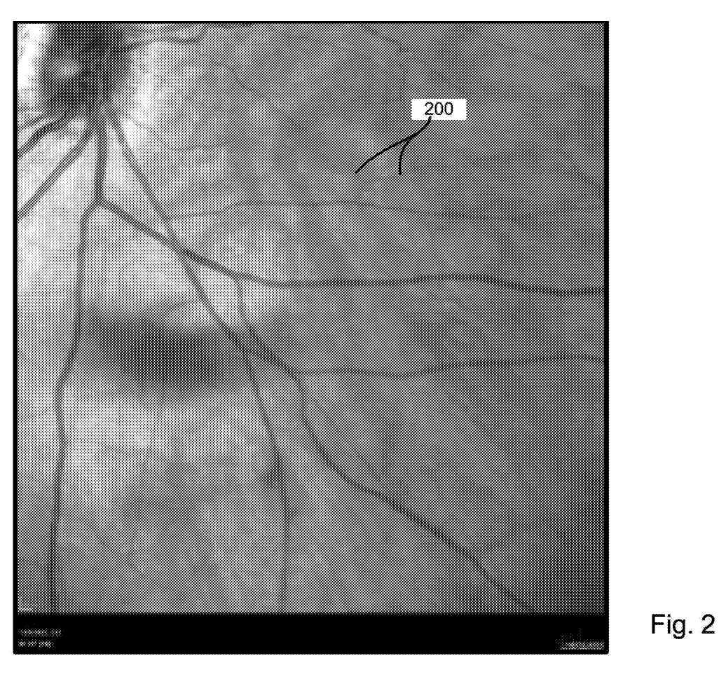

FIG. 2 illustrates an image of a retina that may be captured using the ophthalmic microscope 102. The illustrated image and other images discussed herein are two-dimensional images. However, a pair of binocular images may be used to generate a three-dimensional or volumetric image that may be processed in a like manner.

The image may include representations 200 of blood vessels of the retina. Although some blood vessels are large and may exhibit color enabling distinguishing between veins and arteries, others may be small and the representations 200 thereof may be faint. In addition, any bleeding during a treatment may further make it difficult to perceive the representations 200. During a treatment such as retinal laser photocoagulation it may further be difficult to distinguish between treated and untreated blood vessels. The approach described below facilitates the visualization of blood vessels using an ophthalmic microscope that enables a surgeon to more readily perceive blood vessels, to distinguish between veins and arteries, and to distinguish between treated and untreated blood vessels.

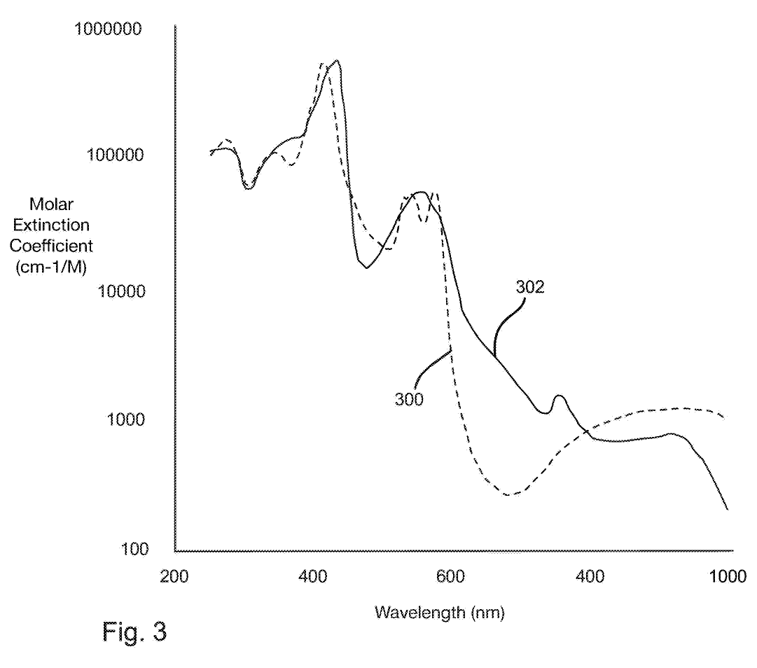

FIG. 3 illustrates a plot of absorption (extinction ratio) with respect to wavelength for oxygenated hemoglobin (plot 300) and de-oxygenated hemoglobin (plot 302). As is apparent, there are isosbestic wavelength where the oxygenated hemoglobin and de-oxygenated hemoglobin have the same absorption and there are also wavelengths at which there are large differences in absorption particularly in the red to infrared spectrum (600 to 1000 nanometers (nm)).

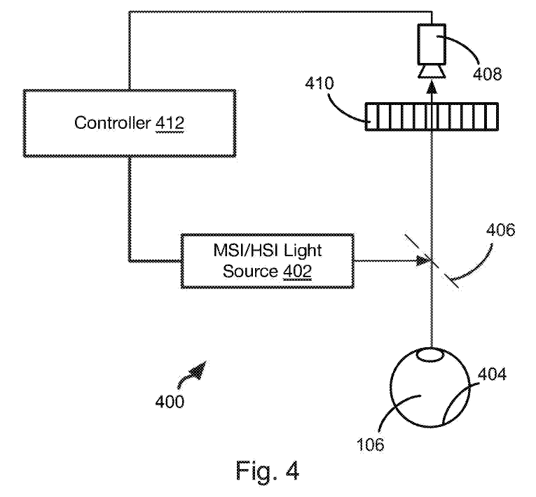

FIG. 4 illustrates a system 400 that uses the different absorption spectra of oxygenated and de-oxygenated hemoglobin to facilitate the visualization of blood vessels. The system 400 may include a light source 402. The light source 402 may be configured to selectively emit light in specific wavelength bands which may correspond to the isosbestic wavelength point such as 586nm and 808nm and . The light source 402 may be a light source as known in the art for performing multi-spectral imaging (MSI), hyper-spectral imaging (HSI), or other imaging modality.

The light source 402 may include a separate light emitting device and/or separate filters to enable control of the wavelength bands. In some embodiments, each wavelength band is less than 20 nm wide, less than 15 nm wide, or less than or equal to 10 nm wide. In some embodiments, at least two images are captured with the retina 404 illuminated with a different wavelength band when each image of the two images is captured. However, images may be captured for more wavelength bands.

The wavelength bands may be selected based on the absorption spectra of oxygenated and de-oxygenated hemoglobin. For example, wavelength bands may be emitted at or near isosbestic wavelengths for oxygenated and de-oxygenated hemoglobin, e.g., where the absorption spectra are substantially (e.g., within 3 dB) the same. The isosbestic wavelength bands may be centered on, for example, 339±5nm, 391±5nm, 423±5nm, 453±5nm, 500±5nm, 529±5nm, 545±5nm, 571±5nm, 586±5nm, and 808±5nm. As used herein, a wavelength band may include a band of wavelengths for which the emitted amplitude of wavelengths outside of the wavelength band is at least 3 dB less than the peak emitted amplitude within the wavelength band.

The wavelength bands may include a wavelength band including the absorption peak for oxygenated hemoglobin, e.g., centered on 414±5nm. The wavelength bands may include a wavelength band including the absorption peak for de-oxygenated hemoglobin, e.g., centered on 433±5nm.

The wavelength bands may include wavelength bands at which absorption for oxygenated and de-oxygenated hemoglobin are very different, e.g., some or all of centered on 370±5nm, 414±5nm, 436±5nm, 458±5nm, and 464±5nm.

The light source 402 may be used with combining optics 406, such as a beam splitter that directs at least a portion of light emitted by the light source 402 to the eye 106 and directs at least a portion of light reflected from the eye 106 to a camera 408 of the ophthalmic microscope 102.

In some embodiments, a filter wheel 410 or other set of selectable filters are used in combination with, or in place of, the light source 402 configured to generate light in specific wavelength bands as defined above. For example, a controller 412 may be coupled to the light source 402 and the filter wheel 410. The controller 412 may configure the light source 402 to emit light in a wavelength band and configure the filter wheel 410 such that light reflected from the eye 106 passes through a passband filter before reaching the camera 408, the passband filter having a passband that includes the wavelength band and reduces the amount of light outside the wavelength band that reaches the camera 408. For example, a passband filter may be a filter having a 3dB bandwidth of less than 20 nm, less than 15 nm, or less than or equal to 10 nm. Alternatively, the light source 402 may include a broadband light source (e.g., a 3dB bandwidth of greater than 20, 40, 100, or 200 nm) and passband filters of the filter wheel 410 alone are used to capture images for a wavelength band.

As used herein the phrase “image for a wavelength band” may be understood as meaning any of:

an image captured by the camera 408 with the retina 404 illuminated by the light source 402 emitting light having the wavelength band.

an image captured by the camera 408 with the retina 404 illuminated with a broadband light source 402 and with light reaching the camera 408 passing through a passband filter for the wavelength band.

an image captured by the camera 408 with both of (a) the retina 404 illuminated by the light source 402 emitting light having the wavelength band and (b) light reaching the camera 408 passing through a passband filter for the wavelength band.

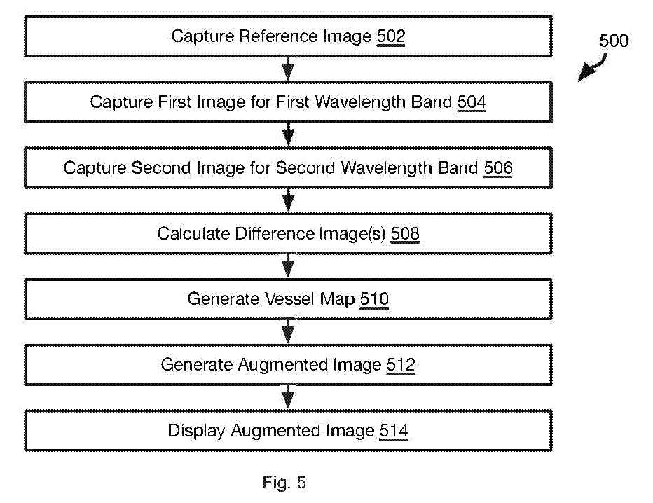

FIG. 5 illustrates a method 500 that may be performed by the controller 412 using the camera 408, light source 402, and possibly a filter wheel 10.

The method 500 may include capturing a reference image at step 502. A reference image may be captured with a broadband light source and without filtering by the filter wheel 10 or with a broadband filter as “broadband” is defined above. The reference image may also be an image captured for an isosbestic wavelength band.

The method 500 may include capturing a first image for a first wavelength band at step 504 and capturing a second image for a second wavelength band at step 506. For example, the first wavelength band may be a wavelength band for which oxygenated hemoglobin has higher absorption relative to de-oxygenated hemoglobin. The second wavelength band may be a wavelength band for which de-oxygenated hemoglobin has higher absorption relative to oxygenated hemoglobin.

The method 500 may include calculating, at step 508, one or more difference images. For example, a first difference image calculated at step 508 may be a difference between the reference image and the first image. A second difference image calculate at step 508 may be a difference between the reference image and the second image. The first difference image will therefore highlight arteries whereas the second difference image will highlight veins.

The calculation of step 508 may be more complex. In particular, any calculating used to calculate blood oxygenation based on the absorption spectra of oxygenated and de-oxygenated hemoglobin may be used. For example, for a given pixel position, intensity values in the reference image, first image, and second image for that pixel position may be used according to any calculation known in the art for estimating blood oxygenation at that position. In still other embodiments, additional images may be captured for one or more other wavelength bands and pixel intensities for a given pixel position for the reference image and three, four, or more other images for three, four, or more wavelength bands may be used to calculate blood oxygenation for that pixel position using any calculation known in the art for calculating blood oxygenation based on absorption spectra of oxygenated and de-oxygenated hemoglobin.

The calculation of step 508 may be using the approach described in the following references, both of which are hereby incorporated herein by reference in their entirety:

Linsenmeier RA, Zhang HF. Retinal oxygen: from animals to humans. Progress in Retinal and Eye Research. 2017;58:115-51.

Garg AK, Knight D, Lando L, Chao DL. Advances in Retinal Oximetry. Transl. Vis. Sci. Technol. 2021;105

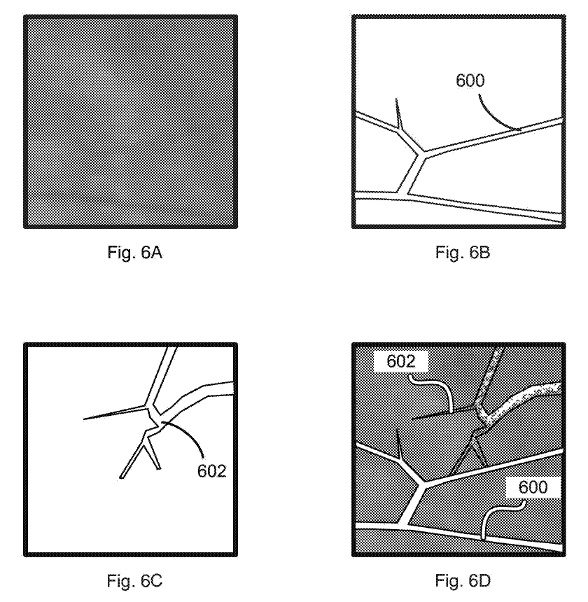

The method 500 may include generating a vessel map at step 510. For example, FIG. 6A illustrates a reference image. FIG. 6B shows a representation 600 of an artery may be identified from some or all of the reference image, first difference image, and second difference image. For example, first pixels identified as corresponding to oxygenated blood (e.g., oxygenation above 85, 88, 90, or 92 percent) may be identified from some or all of the reference image, first difference image, and second difference image such that the first pixels constitute representations 600 of arteries of the retina 404. FIG. 6C shows a representation of a vein resulting from step 508. For example, second pixels may be identified as corresponding to de-oxygenated blood (e.g., oxygenation below 90, 88, 86, or 83 percent) from some or all of the reference image, first difference image, and second difference image such that the second pixels constitute representations 602 of veins of the retina 404.

Step 510 may further include omitting pixels that do not correspond to veins or arteries from the representations 600, 602. For example, pixels that do not exhibit a change in intensity greater than a threshold relative to the reference image in either of the first difference image or the second difference image may be deemed not to correspond to veins or arteries. This process may include vessel extraction algorithm such as an artificial intelligence based method.

The method 500 may include generating, at step 512, an augmented image. For example, an image of the retina captured using broadband lighting and without filtering or with broadband filtering (“the display image”) may have vessel maps from step 510 superimposed thereon to obtain the augmented image. For example, as shown in FIG. 6D, representations 600 of arteries and representations 602 of veins may be superimposed on the display image. The representations 600, 602 may be visually distinguished from one another, such as by being different colors, different fill patterns, or other visually perceptible attribute.

The augmented image may be displayed at step 514, such as one or both display devices 118, 120 or on a display internal to the ophthalmic microscope 102.

The augmented image has the advantage of clearly showing blood vessels, and visually distinguishing veins and arteries. Blood vessels that are not conducting oxygenated or de-oxygenated blood may be omitted from the vessel map and therefore not be highlighted in the augmented image. Accordingly, untreated blood vessels are readily distinguished from treated blood vessels that are no longer conducting blood.

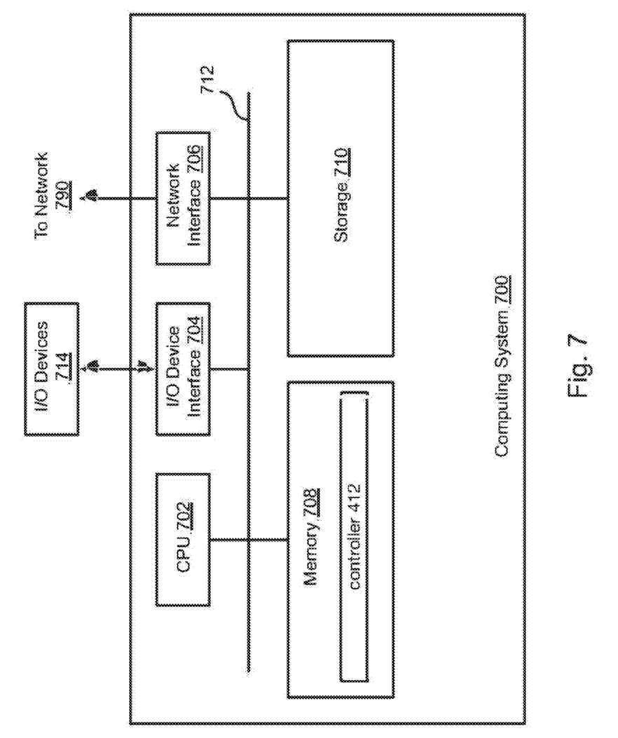

FIG. 7 illustrates an example computing system 700. The ophthalmic microscope 102 and the display devices 118, 120 may incorporate a computing device having some or all of the attributes of the computing system 700.

As shown, computing system 700 includes a central processing unit (CPU) 702, one or more I/O device interfaces 704, which may allow for the connection of various I/O devices 714 (e.g., keyboards, displays, mouse devices, pen input, etc.) to computing system 700, network interface 706 through which computing system 700 is connected to network 790, a memory 708, storage 710, and an interconnect 712.

CPU 702 may retrieve and execute programming instructions stored in the memory 708. Similarly, CPU 702 may retrieve and store application data residing in the memory 708. The interconnect 712 transmits programming instructions and application data, among CPU 702, I/O device interface 704, network interface 706, memory 708, and storage 710. CPU 702 is included to be representative of a single CPU, multiple CPUs, a single CPU having multiple processing cores, and the like.

Memory 708 is representative of a volatile memory, such as a random access memory, and/or a nonvolatile memory, such as nonvolatile random access memory, phase change random access memory, or the like. As shown, memory 708 may store executable code implementing the controller 412.

Storage 710 may be non-volatile memory, such as a disk drive, solid state drive, or a collection of storage devices distributed across multiple storage systems. Storage 710 may optionally store a reference image 722 and a treatment plan 724 for an ophthalmic treatment as defined above.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic visualization system comprising:
an ophthalmic microscope configured to capture video of an eye of a patient; and
a computer system coupled to the ophthalmic microscope and configured to:
receive a first image from the ophthalmic microscope for a first wavelength band having a first width of less than 20 nanometers;
receive a second image from the ophthalmic microscope for a second wavelength band having a second width of less than 20 nanometers;
generate a vessel map of a retina of the eye of the patient according to the first image and the second image, the vessel map including representations of blood vessels of the retina; and
display the vessel map on a display device.

2. The system of claim 1, wherein oxygenated and de-oxygenated hemoglobin have similar absorption in the first wavelength band and different absorption in the second wavelength band.

3. The system of claim 1, wherein oxygenated and de-oxygenated hemoglobin have different absorption in the first wavelength band and in the second wavelength band.

4. The system of claim 1, wherein the vessel map includes representations of veins and arteries of the retina that are visually distinct from one another.

5. The system of claim 1, wherein the computer system is configured to:
receive the first image from the ophthalmic microscope for the first wavelength band by receiving the first image with the retina being illuminated with first light having the first wavelength band; and
receive the second image from the ophthalmic microscope for the second wavelength band by receiving the second image with the retina being illuminated with second light having the second wavelength band.

6. The system of claim 1, wherein the computer system is configured to:
receive the first image from the ophthalmic microscope for the first wavelength band by receiving the first image with first light reflected from the retina being filtered by a first filter with a passband including the first wavelength band; and
receive the second image from the ophthalmic microscope for the second wavelength band by receiving the second image with second light reflected from the retina being filtered by a second filter with a passband including the second wavelength band.

7. The system of claim 1, wherein the computer system is further configured:
receive a reference image from the ophthalmic microscope for a third wavelength band having a third width of less than 20 nanometers; and
generate the vessel map of the retina according to the first image, the second image, and the reference image, wherein:
the third wavelength band is an isosbestic wavelength band, and
oxygenated and de-oxygenated hemoglobin have different absorption in the first wavelength band and the second wavelength band.

8. The system of claim 7, wherein the third wavelength band is centered on one of 339±5nm, 391±5nm, 423±5nm, 453±5nm, 500±5nm, 529±5nm, 545±5nm, 571±5nm, 585±5nm, and 799±5nm.

9. The system of claim 8, wherein the first wavelength band is centered on 414±5nm and the second wavelength band is centered on 433±5nm.

10. The system of claim 1, wherein the computer system is configured to display the vessel map superimposed on an image of the retina.

11. A method comprising:
receiving, by a computer system, from an ophthalmic microscope, a first image from the ophthalmic microscope for a first wavelength band having a first width of less than 20 nanometers;
receiving, by the computer system, from the ophthalmic microscope, a second image from the ophthalmic microscope for a second wavelength band having a second width of less than 20 nanometers;
generating, by the computer system, a vessel map of a retina of an eye of a patient according to the first image and the second image, the vessel map including representations of blood vessels of the retina; and
display the vessel map on a display device.

12. The method of claim 11, wherein oxygenated and de-oxygenated hemoglobin have similar absorption in the first wavelength band and different absorption in the second wavelength band.

13. The method of claim 11, wherein oxygenated and de-oxygenated hemoglobin have different absorption in the first wavelength band and the second wavelength band.

14. The method of claim 11, wherein the vessel map includes representations of veins and arteries of the retina that are visually distinct from one another.

15. The method of claim 11, further comprising:
receiving, by the computer system, the first image from the ophthalmic microscope for the first wavelength band by receiving the first image with the retina being illuminated with first light having the first wavelength band; and
receiving, by the computer system, the second image from the ophthalmic microscope for the second wavelength band by receiving the second image with the retina being illuminated with second light having the second wavelength band.

16. The method of claim 11, further comprising:
receiving, by the computer system, the first image from the ophthalmic microscope for the first wavelength band by receiving the first image with first light reflected from the retina being filtered by a first filter with a passband including the first wavelength band; and
receiving, by the computer system, the second image from the ophthalmic microscope for the second wavelength band by receiving the second image with second light reflected from the retina being filtered by a second filter with a passband including the second wavelength band.

17. The method of claim 11, further comprising:
receiving, by the computer system, a reference image from the ophthalmic microscope for a third wavelength band having a third width of less than 20 nanometers; and
generating, by the computer system, the vessel map of the retina according to the first image, second image, and reference image, wherein:
the third wavelength band is an isosbestic wavelength band, and
oxygenated and de-oxygenated hemoglobin have different absorption in the first wavelength band and the second wavelength band.

18. The method of claim 17, wherein the third wavelength band is centered on one of 339±5nm, 391±5nm, 423±5nm, 453±5nm, 500±5nm, 529±5nm, 545±5nm, 571±5nm, 585±5nm, and 799±5nm.

19. The method of claim 18, wherein the first wavelength band is centered on 414±5nm and the second wavelength band is centered on 433±5nm.

20. The method of claim 11, further comprising displaying, by the computer system, the vessel map superimposed on an image of the retina.
